(19) United States
(12) Patent Application Publication
(10) Pub. No.: US 2008/0085318 A1
(43) Pub. Date: Apr. 10, 2008

(54) Coated compositions and methods for preparing same

(76) Inventors: S. Rao Cherukuri, Vienna, VA (US); Revanth Babu Mutyala, Frederick, MD (US); Mayur Phadke, Sterling, VA (US)

Correspondence Address: RICHARD R. MUCCINO, 758 SPRINGFIELD AVENUE, SUMMIT, NJ, 07901, US

(21) Appl. No.: 11/904,760

(22) Filed: Sep. 28, 2007

Related U.S. Application Data
(63) Continuation-in-part of application No. 11/458,050, filed on Jul. 17, 2006, now abandoned.
(60) Provisional application No. 60/699,568, filed on Jul. 16, 2005.

(51) Int. Cl. A61K 9/50; A61K 31/135; A61K 31/19; A61P 43/00; A61K 31/60

(52) U.S. Cl. 424/493; 424/497; 514/165; 514/568; 514/646

(57) ABSTRACT

The present invention provides methods for preparing a coated pharmaceutical composition comprising the steps of a) providing a pharmaceutically active agent or a nutraceutical; b) providing a solution or a dispersion of a polyvinyl-based, cellulose-based, acrylate-based, or shellac polymeric coating composition, and mixtures thereof, in a solvent selected from the group consisting of C₁-C₃ alcohols, acetone, and ethyl acetate; and c) coating the pharmaceutical active agent or nutraceutical from step (a) with the coating composition from step (b) in a fluid bed processor by spraying the coating solution from a top spray position downwardly onto the pharmaceutical active agent or nutraceutical to provide the coated pharmaceutical composition. The polymer from the coating composition is present in the coated pharmaceutical composition in an amount from about 1% w/w to about 15% w/w on a dry weight basis.

### PRIORITY DATA

This continuation-in-part application claims priority from U.S. patent application Ser. No. 11/458,050, filed on Jul. 17, 2006, which in turn claims priority from U.S. provisional patent application Ser. No. 60/699,568, filed on Jul. 16, 2005.

### FIELD OF THE INVENTION

The present invention provides methods for preparing a coated pharmaceutical composition comprising the steps of a) providing a pharmaceutically active agent or a nutraceutical; b) providing a solution or a dispersion of a polyvinyl-based, cellulose-based, acrylate-based, or shellac polymeric coating composition, and mixtures thereof, in a solvent selected from the group consisting of C₁-C₃ alcohols, acetone, and ethyl acetate; and c) coating the pharmaceutical active agent or nutraceutical from step (a) with the coating composition from step (b) in a fluid bed processor by spraying the coating solution from a top spray position downwardly onto the pharmaceutical active agent or nutraceutical to provide the coated pharmaceutical composition. The polymer from the coating composition is present in the coated pharmaceutical composition in an amount from about 1% w/w to about 15% w/w on a dry weight basis.

### BACKGROUND OF THE INVENTION

A typical fluid bed dryer, such as a “Wurster” type, comprises a cylindrical outer vessel having a perforated floor through which a heated gas passes upwardly to heat and fluidize a batch of tablets or particles (substrates). A concentric, open-ended inner cylinder is suspended above the center of the perforated floor of the outer vessel. A spray nozzle centered beneath the inner cylinder sprays a coating solution (generally a polymeric solution or dispersion) upwardly into the inner cylinder as the fluidized materials pass upwardly through the spray in the inner cylinder. The particles circulate upwardly through the center of the inner cylinder and downwardly between the inner and outer cylinder. The air that fluidizes the particles also serves to vaporize the water causing the composition to deposit as a film or coating onto the surface of each particle. After repeated passes through the coating zone in an inner cylinder, a sufficient thickness of coating solution accumulates and coalesces over the entire surface of each particle to coat the particle.

U.S. Pat. No. 7,192,608 (Ochiai et al.) discloses a method for making a drug granule comprising a granulation step of spraying a solution of a water-soluble drug on a crystal of the water-soluble drug in the absence of binder in a rotary fluidized bed granulate coating apparatus. U.S. Pat. No. 6,465,009 (Liu et al.) discloses a process for producing a pharmaceutical tablet comprising granulating a formulation comprising a water-soluble polyvinylpyrrolidone and an active ingredient, wherein no organic solvents are included in the formulation, and compressing the product of the granulation into a tablet form. U.S. Pat. No. 6,312,521 (Lee et al. '521.) discloses an apparatus in an upward flowing fluidized bed granulator with a screen positioned across the bottom of the granulator. U.S. Pat. No. 6,264,983 (Upadhyay) discloses a process for producing a directly compressible acetaminophen granulation composition. U.S. Pat. No. 5,718,764 (Walter) discloses an apparatus for coating solid particles comprising a housing including gas-guiding walls to provide a swirling movement. U.S. Pat. No. 5,589,267 (Delwel et al. '267) discloses an encapsulated particle for use in liquid cleaning compositions, which is stable in an alkaline environment and exhibits a volume % compressibility of 20 or less at 300° C. U.S. Pat. No. 5,258,132 (Kamel et al.) discloses a wax encapsulated core material particle for use in liquid cleaning compositions prepared with a Wurster fluid bed dryer. U.S. Pat. No. 5,236,503 (Jones) discloses a fluidized bed coater and shields the spray-discharging nozzle to prevent entry of particles into the spray pattern. U.S. Pat. No. 4,875,435 (Jan et al.) discloses a fluidized bed apparatus for drying particles for directing a flow of air axially and an air inlet directing a circumferential flow of air into the chamber to produce an orbital or swirling motion. U.S. Pat. No. 4,335,676 (Debayeux et al.) discloses a device for introducing a gaseous flow stream in an apparatus for granulating or coating particles that provides a more homogenous gaseous flow stream below the bed and avoids the formation of big agglomerated particles in the bed. U.S. Pat. No. 4,237,814 (Ormós et al.) discloses a multi-cell fluidization apparatus wherein a granulate can be introduced in a continuous operation, in one single fluidization apparatus from one or more, dry or wet starting material(s) to be granulated and coated, respectively, with the possibility of controlling size distribution and physical features of the granulates. U.S. Pat. No. 4,115,929 (Staub et al.) discloses a gas flow distributor for fluidizing a bed of particulate material, which minimizes pressure drop at high superficial gas velocities, while maintaining good particle retention, low component erosion probability, and maximum resistance to hot spot formation. United States patent appln. no. 2007/0178164 (Blau '164) discloses a pharmaceutical composition comprising oxcarbazepine, and a pharmaceutical excipient, wherein the oxcarbazepine in the composition has a broad particle size distribution. United States patent appln. no. 2007/0141071 (Ayres) discloses a method of coating a pharmaceutical substrate wherein the substrate can be submerged, mixed, covered with a molten coating material in which melted coating material is sprayed onto a bed of substrate fluidized in a column of air in a Wurster column. United States patent appln. no. 2006/0153912 (Habich et al.) discloses solid choline ascorbate formulations with reduced sensitivity to external stress factors that do not deliquesce on storage under standard conditions in moist ambient air. United States patent appln. no. 2004/0241252 (Abney et al.) discloses a process modification wherein lithium carboxymethyl cellulose granulation is removed from a fluid bed, solubilized, and then top sprayed on the remaining granulation. United States patent appln. no. 2004/0028735 (Kositprapa) discloses an oral controlled release pharmaceutical composition having a water-soluble binder and a water-insoluble binder. United States patent appln. no. 2001/0055648 (Lee et al. '648) discloses a process during which particles are coated in an upward flowing fluidized bed dryer and an insert allows for a more gentle process. WO 95/30735 (Delwel et al. '735) discloses a process of making polyvinyl ether blend encapsulated particles using the Wurster bottom spray mode. EP 1815849 (Blau et al. '849) discloses a method for producing spray-granulated oxcarbazepine by spraying an excipient with a dispersion of oxcarbazepine. EP 0764201 (Nicholson et al.) discloses a wax-encapsulated core material particle and states that particles move randomly in a top spray mode whereas particles move in a well-defined flow pattern in the bottom spray mode.

Accordingly, there is a need for methods for preparing coated pharmaceutical compositions with improved taste-masking and controlled-release properties, low attrition rates, high coating efficiencies, and improved particle size.

### SUMMARY OF THE INVENTION

The present invention provides methods for preparing a coated pharmaceutical composition comprising the steps of a) providing a pharmaceutically active agent or a nutraceutical; b) providing a solution or a dispersion of a polyvinyl-based, cellulose-based, acrylate-based, or shellac polymeric coating composition, and mixtures thereof, in a solvent selected from the group consisting of C₁-C₃ alcohols, acetone, and ethyl acetate; and c) coating the pharmaceutical active agent or nutraceutical from step (a) with the coating composition from step (b) in a fluid bed processor by spraying the coating solution from a top spray position downwardly onto the pharmaceutical active agent or nutraceutical to provide the coated pharmaceutical composition. The polymer from the coating composition is present in the coated pharmaceutical composition in an amount from about 1% w/w to about 15% w/w on a dry weight basis.

### DETAILED DESCRIPTION OF INVENTION

As used herein, the following terms have the given meanings:

The terms “a,” “an,” and, “the” include plural references unless the context clearly dictates otherwise. Thus, for example, reference to “a drug” includes reference to one or more of such drugs, and reference to “an excipient” includes reference' to one or more of such excipients.

The term “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. The term “drug” is expressly encompassed by the present definition as many drugs and prodrugs are known to have specific physiologic activities. These terms of art are well known in the pharmaceutical and medicinal arts.

The term “about” is used to provide flexibility to a numerical range endpoint by providing that a given value may be “a little above” or “a little below” the endpoint.

The term “admixed” means that the drug and/or other ingredients can be dissolved, dispersed, or suspended in the carrier. In some cases, the drug may be uniformly admixed in the carrier.

The term “attrition rate” refers to the rate at which, or the amount of particles or other articles being coated in a fluid dryer become reduced in size due to frictional/shear forces in the fluidization process to a smaller size that is unacceptable and are therefore effectively lost from further in making a pharmaceutical or nutritional supplement formulation.

The term “blending” refers to intermingling different varieties or grades of constituents such that the separate constituents cannot be distinguished

The term “coating” refers to a method for applying a substantially continuous outer layer of a film or a material, called a coating material, to a base material, called a substrate.

The term “coating efficiency” refers to the reduction in the amount of coating material needed to coat a given amount of composition to be coated.

The terms “concentrations”, “amounts”, and other “numerical data” may be expressed or presented herein in a range format. Such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as Well as 1, 2, 3, 4, and 5, individually. This same principle applies to ranges reciting only one numerical value as a minimum or a maximum. Furthermore, such an interpretation should apply regardless of the breadth of the range or the characteristics being described.

The term “drug”, “pharmaceutically active agent,” “active agent,” and “nutraceutical” may be used interchangeably and refers to a substance that has a measurable physiological effect on a subject when administered thereto.

The terms “formulation” and “composition” are used interchangeably and refer to a mixture of two or more compounds, elements, or molecules. In some embodiments the terms “formulation” and “composition” may be used to refer to a mixture of one or more active agents with a carrier or other excipients.

The term “granulating” refers to an intimate mixture of materials, generally numerous particles forming a larger particle.

The term “nutraceutical” is a combined word for “nutrition” and “pharmaceutical” and refers to foods believed to have a medicinal effect on human health. It can also refer to individual chemicals present in common foods. Non-limiting examples of nutraceuticals include phytonutrients, red wine (resveratrol) as an antioxidant, an anticholesterolemic, broccoli (sulforaphane) as a cancer preventative, and soy and clover (isoflavonoids) to improve arterial health in women, flavinoids, and other antioxidants such as γ-linolenic acid, β-beta carotene, anthocyanins, etc.

The term “particle size” refers to the diameter of an individual granular material. Particle sizes are often measured in microns, which are micrometers or one millionth of a meter.

The term “pharmaceutically acceptable,” such as pharmaceutically acceptable carriers, excipients, etc., means pharmacologically acceptable and substantially non-toxic to the subject to which the particular compound is administered.

The term “pharmaceutically acceptable carrier” and “carrier” may be used interchangeably, and refer to any inert and pharmaceutically acceptable material that has substantially no biological activity, and makes up a substantial part of the formulation.

The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hygroscopicity, and solubility of compounds. See, e.g., H. Ansel et. al., Pharmaceutical Dosage Forms and Drug Delivery Systems (6^{th} Ed. 1995) at pp. 196 and 1456-1457.

The terms “plurality of items”, “structural elements”, “compositional elements”, and “materials” may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The term “prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

The term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. A composition that is “substantially free of” an ingredient or element may still actually contain such item as long as there is no measurable effect thereof.

The terms “top spray” and “top spray coating” refer to a process of spraying a coating material from the top of a fluid dryer in a substantially downward direction into the inner cylinder of the fluid bed dryer as fluidized material passes upwardly through the spray in the inner cylinder.

The term “therapeutically effective amount” means an amount of a therapeutically effective compound, or a pharmaceutically acceptable salt thereof, which is effective to treat, prevent, alleviate or ameliorate symptoms of a disease.

The present invention provides methods for preparing a coated pharmaceutical composition comprising the steps of a) providing a pharmaceutically active agent or a nutraceutical; b) providing a solution or a dispersion of a polyvinyl-based, cellulose-based, acrylate-based, or shellac polymeric coating composition, and mixtures thereof, in a solvent selected from the group consisting of C₁-C₃ alcohols, acetone, and ethyl acetate; and c) coating the pharmaceutical active agent or nutraceutical from step (a) with the coating composition from step (b) in a fluid bed processor by spraying the coating solution from a top spray position downwardly onto the pharmaceutical active agent or nutraceutical to provide the coated pharmaceutical composition. The polymer from the coating composition is present in the coated pharmaceutical composition in an amount from about 1% w/w to about 15% w/w on a dry weight basis.

The invention provides methods to prepare coated compositions that will be taste-masked or of a controlled-release nature. The coated compositions are prepared in a fluid bed system wherein the taste-masking or release-controlling composition (the coating material) is top-sprayed instead of bottom-sprayed. Applicant has discovered that the top spraying method results in an increased efficiency in coating, a reduction in attrition rate, and the ability to increase the batch size significantly compared to the bottom-spray method. Generally, any fluid bed dryer that accepts top-spray is suitable for the present invention.

There are many differences between the top spray and bottom spray “Wurster” type fluid bed dryers. In the top spray method, the coating solution is sprayed counter current to the flow of the particles and the method is useful for general coatings, granulations, and dryings. In top spray, there is no column, the average height that each particle has to rise is therefore less, the coating solution spray pressure pushes the material down so that the particles do not rise to great heights, and hence there is less attrition of the active pharmaceutical agent. In top spray, the pattern of the holes in the bottom distribution plate is substantially uniform, there is more twin/triplet formation, there is a difference in the type of the nozzle. Even if the active pharmaceutical agent is powdery, the top filters do not clog readily.

In the bottom spray method, the coating solution is sprayed concurrent to the flow of the particles and the method is particularly suitable for the controlled release of active ingredients. In bottom spray, there is a column in the center of the sprayer, the average height each particle has to rise is therefore greater because of the column, the coating solution spray pressure causes the material to rise to a greater height, and hence there is more attrition of the active pharmaceutical agent. In bottom spray, the holes are big and the pattern of holes is concentrated in the center of the bottom distribution plate to let more air pass through the column, there is less agglomeration, there is a difference in the type of the nozzle. If the active pharmaceutical agent is powdery, the top filters clog readily.

There are many differences in organic solvent spray and aqueous spray in top spraying methods and many differences in organic solvent spray in bottom spraying methods. In organic solvent top spraying methods, the greater volatility of the organic solvents in the present invention results in the use of less energy to dry the particles compared to the use of the energy needed to dry the particles in aqueous top spraying methods. This results in a lower attrition rate in organic solvent top spraying methods.

Because aqueous mixtures are much less volatile than the organic solvent of the present invention in top spray, much more energy is required to dry the particles, which means more air is required to dry the coated particles and hence the particles have to rise to a greater height, which causes greater attrition.

In organic solvent bottom spraying methods, the average height the particles have to rise is greater causing more attrition especially when the method is scaled up and the active pharmaceutical agent has to rise to a greater height in the fluid bed, which results in a greater attrition rate.

The particle size specifications of the starting active pharmaceutical agent and the resulting coated pharmaceutical composition are also very important. A preferred particle size of the starting active pharmaceutical agent is size A which passes through #60 mesh (250 μm), not less than 10%, and passes through #100 mesh (150 μm), not more than 10%. An optional particle size of the active pharmaceutical agent can be size B which passes through #40 mesh (420 μm), not less than 10%, and passes through #100 mesh (150 μm), not more than 10%. The finished product specifications using active pharmaceutical agents having the preferred particle size A pass through #40 mesh (420 μm), not less than 10%, and pass through #100 mesh (150 μm), not more than 10%.

The particle size of the active pharmaceutical agent is very important. A fully taste masked active ingredient to be used in an orally disintegrating tablet formulation (ODT) must have no bitter/obnoxious/bad taste due to the active agent. There must be no grittiness in the mouth due to small particle size of the finished product. If the active pharmaceutical agent is a fine material, a large amount of coating material will be needed to fully taste mask the powder/fine material. Also the process time of the encapsulation will be increased. Other process issues include clogging of the top filters of the fluid bed coater. Also the coated product is more of granular nature, which can cause content uniformity problems in the blend of the orally disintegrating tablet formulation, which can cause problems with mouth feel in the formulations. If the particle size of the active pharmaceutical agent is coarser, the orally disintegrating tablet formulation will cause grittiness in the mouth due to the large particle size of the finished product.

The top spray coating method of the present invention employing non-aqueous solvents may be used to coat both water-soluble and water-insoluble. pharmaceutically active agents. Water-soluble pharmaceutically active agents are difficult materials to encapsulate or coat with aqueous polymeric dispersions because they generally form lumps. Also many polymers are soluble in the solvents of the present invention and are not water-soluble and so special additives are often required such as pH modifiers and surfactants to form aqueous polymeric dispersions for encapsulation. High processing temperatures are also required when coating with aqueous coating compositions to evaporate water. Only low processing temperatures are required when coating with solvent coating compositions to evaporate the more volatile solvents of the present invention. These low processing temperatures are especially helpful when coating low temperature melting pharmaceutically active agents.

The pharmaceutically active agent in the present invention may be selected from a wide variety of pharmaceutically active agents especially those agents with a strong or otherwise unpleasant taste that must be masked or would benefit being in a controlled-release composition. In one embodiment, the pharmaceutically active agent is water-soluble. In another embodiment, the pharmaceutically active agent is water-in soluble. For example, the pharmaceutically active agent to be coated may be an analgesic, an anti-inflammatory drug, or other bitter-tasting drug. In one embodiment, the pharmaceutically active agent is selected from the group consisting of acetaminophen, aspirin, ibuprofen, and an antibiotic. In a specific embodiment, the pharmaceutically active agent is acetaminophen. In another specific embodiment, the pharmaceutically active agent is aspirin. In yet another specific embodiment, the pharmaceutically active agent is ibuprofen. In yet another specific embodiment, the pharmaceutically active agent is an antibiotic. Illustrative antibiotics include azithromycin and clarithromycin. In yet another embodiment, the pharmaceutically active agent is selected from the group consisting of: ibuprofen, naproxen, codeine, hydrocodone, morphine, and mesalamine.

The nutraceutical in the present invention may be selected from a wide variety of nutraceutical agents especially those agents with a strong or otherwise unpleasant taste that must be masked or would benefit being in a controlled-release composition. For example, the nutraceutical agent to be coated may be a bitter-tasting nutraceutical. Non-limiting examples of nutraceuticals include phytonutrients, red wine (resveratrol) as an antioxidant, an anticholesterolemic, broccoli (sulforaphane) as a cancer preventative, and soy and clover (isoflavonoids) to improve arterial health in women, flavinoids, and other antioxidants such as γ-linolenic acid, β-beta carotene, anthocyanins, etc. Preferred nutraceuticals may be selected from the group consisting of phytonutrients, antioxidants, anticholesterolemics, flavinoids, and isoflavonoids.

The solution or a dispersion of a polymeric coating composition in the present invention may be selected from a wide variety of polymeric coating compositions. Preferred polymeric coating compositions are solutions or dispersions of a polyvinyl-based, cellulose-based, acrylate-based, or shellac polymeric coating composition, and mixtures thereof. Preferred polyvinyl-based polymers include polyvinyl acetate, polyvinyl alcohol and polyethylene glycol co-polymers, polyethylene glycol, polyvinyl acetate phthalate etc. Preferred cellulose-based polymers include ethyl cellulose or hydroxypropylmethyl cellulose, povidone, hydroxylpropyl cellulose, hydroxylethyl cellulose, ethyl cellulose dispersion, carboxy methylcellulose, sodium carboxymethyl cellulose, cellulose acetate phthalate, and hydroxylpropyl methyl cellulose phthalate. Preferred acrylate based polymers include methylmethacrylates and ethylmethacrylates, methacrylic acid dispersions, acrylic acid and their copolymers, and anionic polymers of methacrylic acid and methacrylates with a —COOH group (Eudragit® L100-55, manufactured by Rohm and Haas of Philadelphia, Pa.). Shellac polymeric coating compositions may also be employed. Because of its alkaline properties, shellac polymeric coating compositions may be used for a timed enteric or colonic release. Polyvinyl acetate is a preferred polymer.

The solvent in the polymeric coating composition is a non-aqueous solvent selected from the group consisting of C₁-C₃ alcohols, acetone, and ethyl acetate. C₁-C₃ alcohols comprise methanol, ethanol, n-propanol, and isopropanol. The preferred non-aqueous solvent is isopropanol. Optionally, a quantity of 5-10% water may be added to the non-aqueous solvent of the polymeric coating composition to improve solubility, coating properties, or other properties.

In general, the polymer is present in the coated pharmaceutical composition in an amount from about 1% w/w to about 15% w/w on a dry weight basis, preferably in an amount from about 5% w/w to about 15% w/w on a dry weight basis.

In a preferred embodiment, the solution or a dispersion of a polymeric coating composition comprises ethyl cellulose in isopropanol at a concentration ranging from about 1% w/w to about 40% w/w, preferably from about 10% w/w to about 30% w/w.

By using the top spray method of the present invention, the process produces coated pharmaceutical compositions that are uniform or substantially uniform. The top spray method reduces the attrition rate, the rate at which, or the amount of particles or other articles being coated in a fluid dryer become reduced in size due to frictional/shear forces in the fluidization process to a smaller size that is unacceptable and are therefore effectively lost from further in making a pharmaceutical or nutritional supplement formulation. The top-spray process reduces attrition rate to acceptable levels, for example, to less than about 20% or preferably, to less than about 15% or even more preferably, to less than about 10%, and even more preferably, to less than about 5%.

The top-spray method of the present invention provides an increased efficiency in coating compared to bottom-spray methods. Coating efficiency refers to the reduction in the amount of polymeric coating composition needed to coat a given amount of pharmaceutically active agent or nutraceutical. For example, the present top-spray method provides a reduction of up to about 25% in the amount of coating material that may be needed, preferably up to about 20%.

In one embodiment, the pharmaceutical composition is in crystalline form. In another embodiment, the composition is an amorphous powder. In yet another embodiment, the composition has been granulated. In one embodiment, the granulation is accomplished using a dry granulation process. In yet another embodiment, the granulation is accomplished using a wet granulation method. In yet another embodiment, the granules or particles of the composition range from about 100 microns to about 1500 microns, preferably from about 100 microns to about 1000 microns, more preferably from about 100 microns to about 600 microns, and most preferably from about 100 microns to about 500 microns.

In some embodiments, the top-spray coating process disclosed herein provides the unexpected advantage that batch sizes can be increased up to about 10 times compared to the bottom-spray methods. This increase in batch size provides significant benefits to the overall processing in the pharmaceutical production, which include, economies of scale, reduced validation times, reduced risk of contamination from batch to batch, reduced health and occupational risks relating to materials transfers, etc. The batch sizes that can be employed typically may range from about 5 kilograms to about 500 kilograms, depending upon the equipment available and the need for a given batch size. However, it is within the ordinary skill in the art to modify the fluid bed dryer equipment to increase the batch size if needed, even up to one ton or more.

The present top-spray coating methods are most suitable for high dose drugs such as those requiring dosages ranging from about 100 to 500 mg or even up to about 800 mg of active agent. However, those drugs that are of low dose such as those requiring from about 1 to 100 mg of the active agent may also be conveniently coated under the present methods.

It has been discovered that the present top-spray coating method has produced several of the above-mentioned advantages such as reduced attrition rate, increased batch-size capability, increased coating efficiency, when a coating composition comprising polyvinyl acetate in isopropanol was been used. The particular drug that was advantageously coated using a polyvinyl acetate in isopropanol dispersion was acetaminophen with a dose of from about 100 mg to about 500 mg.

Coating or blending the pharmaceutically active agent or nutraceutical can be helpful for improving the method process depending upon the property of the active agent. Solvents with fats, waxes, or emulsifiers can provide taste masking or control release properties. Spraying acetylated monoglyceride in isopropyl alcohol (10%) with 0.5% talc as a glidant on the top of pharmaceutically active agent or nutraceutical is also useful or spraying acetylated monoglyceride in isopropyl alcohol with talc as a second coating on the top of first coating is also useful. In the present invention, the fluid bed is maintained at a temperature below the melting point of a fat, wax or emulsifier such that the top spray coating using a solution/dispersion of wax/fat/emulsifier does not require melting the coating material. In one embodiment, the coating does not produce substantially granulation.

The coated pharmaceutical compositions of the present invention can be prepared according to the examples set out below. The examples are presented for purposes of demonstrating, but not limiting, the preparation of the dosage forms of this invention.

### EXAMPLES

### Example 1

Acetaminophen was encapsulated using aqueous coating in a fluid bed processor with primary objective to mask the bitter taste of acetaminophen. Good taste masking was achieved using aqueous coating solution, with top-spray coating. The coating solution was prepared by weighing the ingredients and mixing them in purified water, USP. The compositions of this example are shown in Table 1 below.

TABLE 1

| | | | Quantity (%) |
|---|---|---|---|
| S. No | Generic Name | Supplier | (On dry basis) |
| 1 | Acetaminophen | Granules USA | 88.0% |
| 2 | Polyvinyl acetate | BASF | 13.5% solid |
| | 30% dispersion | | content |
| 3 | Polyvinyl alcohol/ | BASF | 1.5% |
| | Polyethylene glycol | | |
| | graft copolymer | | |
| 4 | Purified Talc | Mineral and Pigment | 0.5% |
| | | Solutions, Inc. | |
| 5 | Propylene glycol | Dow Chemical Co. | 0.25% |
| 6 | Sodium lauryl sulfate | Stepan | 0.074% |
| 7 | Purified water | — | — |
| 8 | Colloidal silicon | Grace | 0.85% |
| | dioxide | | |

Fluid bed processors, UFBM-60 and UFBM-3 were used, depending on the batch size. The processor was run under either the auto or manual mode. Batches of various sizes were prepared, ranging from about 2 to 50 kilos. The blower was run at rpms ranging from about 1200 rpms to 2000 rpms. The blower rpm was varied to minimize the carryover fine particles and to ensure low-pressure drop across the filters. Pump rpm (spray rate, gm/min) was also optimized so that uniform coating was obtained while agglomeration was minimized. The spray rate was changed as needed. The optimal range of pump rpm was between 10 to 25 rpm. Inlet temperature was fixed based on bed temperature. The inlet temperature for aqueous coating ranged from about 40° C. to about 80° C. The bed temperature was based on the type of solvent used for coating solution. The aqueous solutions needed a higher temperature compared to the non-aqueous solutions. The optimized range of bed temperatures for the aqueous operation was 46° C. to 51° C. Atomizing pressure ranged from about 4.0 to 6, kg/cm². Spray nozzles had a diameter ranging from about 0.6 mm to about 1.5 mm. In some cases, about 1.0 mm diameter was used. Wide-angle spray nozzle was also tried. Spray nozzle height was adjusted to suit the material and batch size. Three different positions, low, middle, and top positions were used. In some cases, the top-most position was useful. Particle size analysis was performed using Rotap Sieve shaker. ASTM (American Society for Testing and Materials) sieves of mesh #30, #40, #50 and #80 were used to determine particle size distribution.

### Example 2

The methodology of Example 1 above was used to prepare a top-spray coating composition for taste-masking ibuprofen. The composition of Table 1 was used with the following change. Ibuprofen in comparable quantity was used in place of acetaminophen. The remaining ingredients were adjusted proportionately.

### Example 3

The methodology of Example 1 above was used to prepare a top-spray coating composition for taste-masking aspirin. The composition of Table 1 was used with the following changes. Aspirin in comparable quantity was used in place of acetaminophen and the coating composition was changed to an ethyl cellulose composition in isopropanol in comparable amounts. The remaining ingredients were adjusted proportionately.

### Example 4

The methodology of Example 1 above was used to prepare a top-spray coating composition for taste-masking aspirin. The composition of Table 1 was used with the following changes. Aspirin in comparable quantity was used in place of acetaminophen and the coating composition was changed to an ethyl cellulose composition in isopropanol in comparable amounts. The remaining ingredients were adjusted proportionately.

### Example 5

A quantity of 5.0 kg of ethyl cellulose was dissolved in 48.410 kg of isopropyl alcohol. after the solution was formed, 0.116 kg of polyethylene glycol 600 was dissolved in the solution. A quantity of 0.116 kg of talc was sifted through an ASTM mesh #60 into the solution under continuous stirring to form a uniform suspension. A quantity of 0.250 kg of sodium lauryl sulfate was dissolved in 0.490 kg of purified water and was added to the non-aqueous solution with continuous stirring. A fluidized bed processor with a top spray bowl was warmed to 40° C. The fluidized bed processor was setup with the solution previously prepared. A quantity of 50 kg of acetaminophen of mesh #60 to mesh #100 was weighed and charged into the top spray bowl of the fluidized bed processor. The blower of the fluid bed coater was started and bed was warmed up to 40° C. Coating was started and continued with the following parameters: spray rate at 127 g/min, Inlet temperature: 50° C. to maintain bed temperature: 40±2° C., blower load 70% (about 350 CFM), spray air pressure: 45 psi. A yield of 96.9% was obtained. The total time to coat the material with the solution took about 7 hours. A quantity of 7% agglomerated material was obtained over mesh #40. No attrition was observed. The material did not taste bitter and was found to be satisfactory when used in an orally disintegrating tablet formulation.

### Example 6

A quantity of 5.0 kg of ethyl cellulose was dissolved in 48.410 kg of isopropyl alcohol. After a solution was formed, 0.116 kg of polyethylene glycol 600 was dissolved in the solution. A quantity of 0.116 kg of talc was sifted through an ASTM mesh #60 into the solution with continuous stirring to form a uniform suspension. A quantity of 0.250 kg of sodium lauryl sulfate was dissolved in 0.490 kg of purified water and was added to the non-aqueous solution with continuous stirring. A fluidized bed processor with a Wurster (bottom) spray bowl was warmed to 40° C. The fluidized bed processor was setup with the solution previously prepared. A quantity of 50 kg of acetaminophen of mesh #60 to mesh #100 was weighed and charged into the Wurster spray bowl of the fluidized bed processor. The blower of the fluid bed coater was started and bed was warmed to 40° C. Coating was started and continued with the following parameters: spray rate at 127 g/min, Inlet temperature: 50° C. to maintain bed temperature: 40±2° C., blower load 80% (about 400 CFM), spray air pressure: 45 psi, needle pressure: 40 psi. The total time to coat the material with the solution took about 8 hours. No agglomerated material was obtained over mesh #40, while there was a lot of attrition observed. A yield of 85.6% was obtained because some powder generated from attrition escaped through the filters. The material below mesh #100 was observed to be around 12 kg, which was about 21.6% of the total batch size. The coated material tasted bitter (hence the objective of encapsulation was not achieved) and this material was not suitable for an orally disintegrating tablet formulation.

### Example 7

A quantity of 61 g of Eudragit® L100-55 (an anionic polymer of methacrylic acid and methacrylates with a —COOH group) was dissolved in 960 g of isopropyl alcohol. After a solution was formed, a quantity of 61 g of talc was sifted through an ASTM mesh #60 into the solution with continuous stirring to form a uniform suspension. A quantity of 6.1 g of triethyl citrate was added to the non-aqueous mixture with continuous stirring. A fluidized bed processor with a top spray bowl was warmed to 40° C. The fluidized bed processor was setup with the solution previously prepared. A quantity of 871 g of ibuprofen of mesh #60 to mesh #100 was weighed and charged into the top spray bowl of the fluidized bed processor. The blower of the fluid bed coater was started and bed was warmed to 40° C. Coating was started and continued with the following parameters: spray rate at 3.0 g/min, Inlet temperature: 50° C. to maintain bed temperature: 40±2° C., blower load 1050 RPM, spray air pressure: 1.4 psi to give a coating of 7%. A yield of about 95% was obtained. The total time taken to coat the material with the solution took about 8 hours. A quantity of 1% agglomerated material was obtained over mesh #40. No attrition was observed.

### Example 8

A quantity of 75 g. of ethyl cellulose was dissolved in 1500 mL of isopropyl alcohol. A fluidized bed processor with a top spray bowl was warmed to 35° C. The fluidized bed processor was setup with the solution previously prepared. A quantity of 750 kg of vitamin C of mesh #60 to mesh #100 was weighed and charged into the top spray bowl of the fluidized bed processor. The blower of the fluid bed coater was started and bed was warmed to 35° C. Coating was started and continued with the following parameters: spray rate at 3.6 mL/min, Inlet temperature: 50° C. to maintain bed temperature: 40±2° C., blower at 1100 RPM, spray air pressure: 1.2 psi. A yield of 94% was obtained. The total time to coat the material with the solution took about 7 hours. A quantity of 4% agglomerated material was obtained over mesh #40. No attrition was observed.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A method for preparing a coated pharmaceutical composition comprising the steps of:
a) providing a pharmaceutically active agent or a nutraceutical;
b) providing a solution or a dispersion of a polyvinyl-based, cellulose-based, acrylate-based, or shellac polymeric coating composition, and mixtures thereof, in a solvent selected from the group consisting of C₁-C₃ alcohols, acetone, and ethyl acetate; and
c) coating the pharmaceutical active agent or nutraceutical from step (a) with the coating composition from step (b) in a fluid bed processor by spraying the coating solution from a top spray position downwardly onto the pharmaceutical active agent or nutraceutical to provide the coated pharmaceutical composition;
wherein the polymer from the coating composition is present in the coated pharmaceutical composition in an amount from about 1% w/w to about 15% w/w on a dry weight basis.

2. The method according to claim 1, wherein the pharmaceutically active agent or nutraceutical is water-soluble.

3. The method according to claim 1, wherein the pharmaceutically active agent or nutraceutical is water-insoluble.

4. The method according to claim 1, wherein the pharmaceutically active agent in step (a) is selected from the group consisting of acetaminophen, aspirin, ibuprofen, and an antibiotic.

5. The method according to claim 4, wherein the pharmaceutically active agent is acetaminophen.

6. The method according to claim 4, wherein the pharmaceutically active agent is aspirin.

7. The method according to claim 4, wherein the pharmaceutically active agent is ibuprofen.

8. The method according to claim 4, wherein the pharmaceutically active agent is an antibiotic.

9. The method according to claim 1, wherein the nutraceutical is selected from the group consisting of phytonutrients, antioxidants, anticholesterolemics, flavinoids, and isoflavonoids.

10. The method according to claim 1, wherein the particle size of the pharmaceutically active agent or nutraceutical in step (a) is from about 150 microns to about 420 microns.

11. The method according to claim 1, wherein the polymeric coating composition in step (b) comprises polyvinyl acetate, polyvinyl alcohol and polyethylene glycol co-polymers, polyethylene glycol, polyvinyl acetate phthalate, ethyl cellulose, hydroxy propylmethyl cellulose, povidone, hydroxylpropyl cellulose, hydroxylethyl cellulose, ethyl cellulose dispersion, carboxy methyl cellulose, sodium carboxy methyl cellulose, cellulose acetate phthalate, hydroxylpropyl methyl cellulose phthalate, ethylmethacrylates, methacrylic acid aqueous dispersion, acrylic acid, anionic polymers of methacrylic acid and methacrylates with a —COOH group, and shellac.

12. The method according to claim 1, wherein the polymer from the coating composition is present in the coated pharmaceutical composition in an amount from about 5% w/w to about 15% w/w on a dry weight basis.

13. The method according to claim 1, wherein the polymer from the coating composition comprises polyvinyl acetate present in an amount from about 1% w/w to about 15% w/w on a dry weight basis.

14. The method according to claim 1, wherein the method has an attrition rate of about 15% or less.

15. The method according to claim 1, wherein the method has a coating efficiency of about 20% or less.

16. The method according to claim 1, wherein the solvent in step (b) is isopropanol.

17. The method according to claim 1, wherein the solvent in step (b) further comprises from about 5% to about 10% water.

18. The method according to claim 1, further comprising blending the pharmaceutically active agent or nutraceutical in step (a) with a diluent.

19. The method according to claim 1, further comprising blending the polymeric coating composition in step (b) with a fat, wax or emulsifier.

20. The method according to claim 1, further comprising adding acetylated monoglyceride and talc in isopropanol to the pharmaceutically active agent or nutraceutical in step (a).

21. The method according to claim 1, further comprising adding acetylated monoglyceride and talc in isopropanol as a second coating over the coated pharmaceutical composition.
